(19) United States
(12) Patent Application Publication
(10) Pub. No.: US 2024/0245593 A1
(43) Pub. Date: Jul. 25, 2024

(54) COOLING FORMULATION FOR TOPICAL APPLICATION

(71) Applicant: Cold Sole, LLC, Bala Cynwyd, PA (US)

(72) Inventor: Linda Mullens, Bala Cynwyd, PA (US)

(21) Appl. No.: 18/420,608

(22) Filed: Jan. 23, 2024

Related U.S. Application Data
(60) Provisional application No. 63/440,489, filed on Jan. 23, 2023.

(51) Int. Cl. A61K 8/42; A61K 31/22; A61Q 17/04

(52) U.S. Cl. CPC A61K 8/42; A61K 31/22; A61Q 17/04

(57) ABSTRACT

A cooling formulation having an effective amount of an active cooling agent that modulates TRPM8 receptors, wherein the active cooling agent includes 5-10%, by volume, menthyl ethylamido oxalyate; an effective amount of UV protective agents that provide an SPF of at least 30; and further comprising 5-10% cooling oil.

### CROSS-REFERENCE TO RELATED APPLICATIONS

This application for a utility patent claims the benefit of U.S. Provisional Application No. 63/440,489, filed Jan. 23, 2023.

### BACKGROUND OF THE INVENTION

### Field of the Invention

This invention relates generally to soothing topical formulations, and more particularly to a cooling and anesthetic formulation for topical application.

### Description of Related Art

The prior art teaches topical formulations that include aloe, which has some cooling and soothing properties, and which also provides a small amount of skin protection, typically about 14 SPF. While aloe formulations provide a small amount of each quality, cooling/soothing and SPF protection, these formulations are limited in their effect, and do not provide a combination of strong cooling sensation and adequate SPF protection.

The prior art teaches aloe based topical formulations. However, the prior art does not teach a topical formulation that includes both superior cooling sensations and also significant UC protection (30 SPF of higher). The present invention fulfills these needs and provides further advantages as described in the following summary.

### SUMMARY OF THE INVENTION

The present invention teaches certain benefits in construction and use which give rise to the objectives described below.

The present invention provides a cooling formulation for providing a cooling effect and for protecting from the sun. The cooling formulation comprises an effective amount of an active cooling agent that modulates TRPM8 receptors, wherein the active cooling agent includes 5-10%, by volume, menthyl ethylamido oxalyate; an effective amount of UV protective agents that provide an SPF of at least 30; and further comprising 5-10% cooling oil.

A primary objective of the present invention is to provide a cooling formulation having advantages not taught by the prior art.

Another objective is to provide a cooling formulation that provides active cooling by blocking TRPM8 receptors.

A further objective is to provide protection against UV radiation of at least 30 SPF.

Other features and advantages of the present invention will become apparent from the following more detailed description, taken in conjunction with the accompanying drawings, which illustrate, by way of example, the principles of the invention.

### DETAILED DESCRIPTION OF THE INVENTION

The above-described drawing figures illustrate the invention, a topical formulation that includes an active cooling agent, and also include a UV protective component that provides at least 30 SPF protection from UV radiation. The topical formulation may be applied to the skin of a user to provide superior active cooling, as described in greater detail below. In one embodiment, the topical formulation is in aerosol form that may be sprayed or otherwise applied to a user's skin to protect from discomfort, such as the discomfort of being in a hot environment, walking on hot sand while at the beach, or other similar usages, and also protect the user from skin damage while in this hot and rugged environment.

The cooling formulation includes at least one active cooling agent active to actively cool the skin. The formulation includes an effective amount of an active cooling agent that modulates TRPM8 receptors. For purposes of this application, the term “effective amount” is defined to include a therapeutically effective amount to achieve the desired effect, as determined by one skilled in the art of this field. These agents not only provide a cooling sensation, they also reduce discomfort experienced by the user from a hot environment. This can help to alleviate discomfort experienced in a hot and rugged environment, such as, for example, the beach, or other hot outdoor environments.

Furthermore, the formulation may further include one of more topical anesthetics, which may be used to minimize pain, swelling, and redness in the area. Various topical anesthetic agents available for use are eutectic mixture of local anesthetics, ELA-max, lidocaine, epinephrine, tetracaine, bupivanor, 4% tetracaine, benzocaine, proparacaine, Betacaine-LA, topicaine, and Lidoderm. The anesthetics also have antiseptic properties which further help relieve any discomfort and can also help reduce any potential spread of infection.

In various embodiments, the cooling formulation may further include an effective amount of a cooling oil such as Peppermint Oil, Chamomile Oil, and Clove Leaf Oil to provide a cooling sensation for the user when applied to the skin, which helps relieve pain in the tissues underneath the skin. In one embodiment, approximately 2.0%, 2.0%, and 4.5% are used, respectively. All percentages are of total final volume.

In one embodiment, the total amount of cooling oils is about 5.0-10.0%, in this case about 8.5%. This range of suitable amounts of oils and other agents is helpful to provide the necessary final characteristics of the final topical formulation.

Other suitable oils include Tea Tree Oil, Wintergreen Oil, and other equivalent oils known in the art. Wintergreen Oil contains menthol and menthone, and it provides cooling properties, as well as soothing pain and providing anti-inflammatory properties. Clove oil provides anti-microbial properties as well as pain relief. In some embodiments, the formulation may further include Peppermint Oil, and/or other extracts from mint, such as spearmint and watermint.

In some embodiments, a propellant phase is also included in the formulation, which may include N-Butane, Propane, 1,1,1,3,3-Pentafluoropropane, 1,1,1,2-Tetrafluoroethane (aka norflurane), and Isobutane. In one embodiment, the propellant phase acts as a topical vapocoolant for cooling and numbing the skin. The cooling formulation may further include a pharmaceutically acceptable carrier, stabilizers and preservatives.

The formulation is designed to provide a cooling sensation that will help relieve pain in the tissues of the skin. When applied, the active ingredients which may include an anesthetic, Menthol, and other ingredients noted above create a cooling and numbing effect on the skin.

The formulation also contains Dimethyl Isosorbide, CERAPHYL™ 230, and Dimethicone, which act as moisturizers and help to protect the skin from harsh environments.

In a first embodiment, a set of “Phase A” ingredients are as follows: about 5% N-Pentane, about 75% Ethanol, about 5% CERAPHYL™ 230, about 4.5% PEG-8 Dimethicone, about 2% FRESCOLAT® X-cool, about 2% Gaultheria procumbens (Wintergreen) Leaf Oil, about 2% Chamomilla recutita (Matricaria) Flower Oil, and about 2% Eugenia caryophyllus (Clove) Bud Oil. During manufacturing, in a main vessel, add all Phase A ingredients one at a time with mixing. Next, during a propellant phase, the following ingredients are added: 10% N-Butane, 10% Propane, 20% 1,1,1,3,3-Penafluoropropane, 15% 1,1,1,2-Tetrafluoroethane, and 15% Isobutane. The concentrate is filled into aluminum cans, vacuum crimped, and charged with propellant.

The active ingredients of the second embodiment are: Isobutane, N-Butane, Propane, 1,1,1,3,3-Pentafluoropropane, 1,1,1,2-Tetrafluoroethane, and N-Pentane. The inactive ingredients are: Ethanol, Diisopropyl Adipate, PEG-8 Dimethicone, Eugenia caryophyllus (Clove) Bud Oil, Mentha piperita (Peppermint) Oil, Chamomilla recutita (Matricaria) Flower Oil, and Menthyl Ethylamido Oxalate.

In the current embodiment, about 2% FRESCOLAT® X-cool (menthyl ethylamido oxalyate) is provided to provide active cooling of the user's skin. These ingredients modulate the TRPM8 receptors to promote cooling sensations. While menthyl ethylamido oxalyate is currently preferred, other similar ingredients may be used, such as menthyl lactate, and also methyl glycerin acetal, which also modulate TRPM8 receptors. Other ingredients may be added to block TRPV1 receptors to prevent feelings of pain and discomfort.

About 75% Ethanol is provided as a solvent, and provides superior properties. At a 75% concentration, ethanol takes longer to evaporate and this longer contact time means that it is able to penetrate cells more effectively. And 75% ethanol also tends to be cheaper and is less flammable that higher (95%) concentrations.

About 5% CERAPHYL™ 230, is provided to provide a superior feel to the user. Ceraphyl™ 230 is composed of very dry, non-greasy esters, which impart a transient feel during rub out but leave minimal residual after feel. It is frequently used to reduce tackiness and lighten the feel of heavier emollients.

About 4.5% PEG-8 Dimethicone, is provided to improve skin feel, detackify ingredients in formulas, and add additional moisturization.

In the second embodiment, a set of “Phase A” ingredients are as follows: about 6% N-Pentane, about 75% Ethanol, about 5% CERAPHYL™ 230, about 4.5% PEG-8 Dimethicone, about 2% FRESCOLAT® X-cool, about 3% Gaultheria procumbens (Wintergreen) Leaf Oil, about 2% Chamomilla recutita (Matricaria) Flower Oil, about 2% Eugenia caryophyllus (Clove) Bud Oil, and about 0.05% Melaleuca alternifolia (Tea Tree) Leaf Oil. During manufacturing, in a main vessel, add all Phase A ingredients one at a time with mixing. Next, during a propellant phase, the following ingredients are added: 10% N-Butane, 10% Propane, 20% 1,1,1,3,3-Penafluoropropane, 15% 1,1,1,2-Tetrafluoroethane, and 15% Isobutane. The concentrate is filled into aluminum cans, vacuum crimped, and charged with propellant.

The active ingredients of the first embodiment are: Isobutane, N-Butane, Propane, 1,1,1,3,3-Pentafluoropropane, 1,1,1,2-Tetrafluoroethane, and N-Pentane. The inactive ingredients are: Ethanol, Diisopropyl Adipate, PEG-8 Dimethicone, Gaultheria procumbens (Wintergreen) Leaf Oil, Menthyl Ethylamido Oxalate, Chamomilla recutita (Matricaria) Flower Oil, Eugenia caryophyllus (Clove) Bud Oil, and Melaleuca alternifolia (Tea Tree) Leaf Oil.

In a third embodiment, the formulation may be sprayed or otherwise applied to a user's feet to protect the feet from discomfort, such as the discomfort of walking on hot sand while at the beach, or other similar usages. In the third embodiment, the cooling formulation includes a numbing agent such as a local anesthetic like Lidocaine (e.g., Xylocaine) and/or Benzocaine (or other ester anesthetic), or an equivalent topical pain reliever, which provide a number of benefits to a user's feet.

The third embodiment of the formulation also contains Dimethyl Isosorbide, Saccharomyces Ferment Filtrate, Albatrellus confluens (Mushroom) Extract, Benzyl Alcohol, Dehydroacetic Acid, Menthyl Ethylamido Oxalate, Barbadensis (Aloe) Leaf Juice, Phenoxyethanol, Polyethylene Glycol 400, and Dimethicone, which act as moisturizers and help to protect the skin from the harsh environment.

This cooling formulation is designed to be sprayed on the feet before walking on hot sand, providing instant relief by providing a cooling sensation that helps to reduce the pain. The formulation can also be used to provide relief to other areas of the body that have been exposed to extreme temperatures and need a cooling sensation.

In each embodiment, this formulation is designed to be applied (i.e., by hand or spraying) to any non-sensitive part of the skin, providing instant relief by providing a cooling sensation that helps to reduce the pain, and also protecting from the sun. The formulation is safe to use and can be applied on a regular basis. The formulation is easy to apply, as it is simple to spray the product onto the skin, wherein it can be absorbed, providing fast relief.

In one embodiment, the formulation is dispensed from an aerosol container which includes a valve for dispensing a desired amount of the formulation. In another embodiment, it may be in the form of a lotion or ointment, which may be manually spread on the feet or other body parts. The formulation may be used as a topical spray, and should not be ingested or used on sensitive areas of the user's skin

The formulation is intended to relieve the pain of skin tissues by providing a cooling sensation. It is suitable for topical application, and contains a variety of active ingredients described above for both cooling and anesthetic qualities. Benefits of the formulation include: 1. A cooling sensation and relief of pain from hot and/or abrasive surfaces. 2. Provides numbing and a topical anesthetic. 3. Can be applied quickly and evenly to the skin. 4. A propellant may be included to ensure the formulation is effectively applied. 5. Easy to transport and store. 6. Relieves discomfort while walking on hot sand.

The formulation includes effective amounts of anesthetic, Menthol, N-Butane, Propane, 1,1,1,3,3-Pentafluoropropane, 1,1,1,2-Tetrafluoroethane, N-Pentane, and Isobutane, wherein upon application to the skin, the active ingredients diffuse onto the outer epidermis providing a cooling sensation for the user when applied to the skin, which helps relieve pain in the tissues underneath the skin.

The title of the present application, and the claims presented, do not limit what may be claimed in the future, based upon and supported by the present application. Furthermore, any features shown in any of the drawings may be combined with any features from any other drawings to form an invention which may be claimed.

As used in this application, the words “a,” “an,” and “one” are defined to include one or more of the referenced item unless specifically stated otherwise. The terms “approximately” and “about” are defined to mean +/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A cooling formulation for providing a cooling effect and for protecting from the sun, said cooling formulation comprising:
an effective amount of an active cooling agent that modulates TRPM8 receptors; and
an effective amount of vapocoolant.

2. The cooling formulation of claim 1, wherein the active cooling agent is present in an amount of about 5.0% of total final volume.

3. The cooling formulation of claim 1, wherein the active cooling agent includes menthyl ethylamido oxalyate.

4. The cooling formulation of claim 1, further comprising 5-10% cooling oil.

5. The cooling formulation of claim 1, further comprising a topical anesthetic.

6. The cooling formulation of claim 1, further comprising an effective amount of UV protective agents that provide an SPF of at least 30.

7. A cooling formulation for providing a cooling effect and for protecting from the sun, said cooling formulation comprising:
an effective amount of an active cooling agent that modulates TRPM8 receptors, wherein the active cooling agent includes 5-10%, by volume, menthyl ethylamido oxalyate;
an effective amount of UV protective agents that provide an SPF of at least 30; and
further comprising 5-10% cooling oil.

8. The cooling formulation of claim 7, further comprising a topical anesthetic.
